(19) United States
(12) Patent Application Publication
(10) Pub. No.: US 2006/0264745 A1
(43) Pub. Date: Nov. 23, 2006

(54) Optical biopsy system with single use needle probe

(76) Inventor: Luiz B. Da Silva, Danville, CA (US)

Correspondence Address: John P. Wooldridge, 114 Honu'ea Pl, Kihei, HI, 96753, US

(21) Appl. No.: 11/375,873

(22) Filed: Mar. 13, 2006

Related U.S. Application Data
(63) Continuation-in-part of application No. 10/803,574, filed on Mar. 17, 2004, now abandoned.
(60) Provisional application No. 60/455,536, filed on Mar. 17, 2003.
(60) Provisional application No. 60/760,196, filed on Jan. 18, 2006.

(51) Int. Cl. A61M 25/00

(52) U.S. Cl. 600/434

(57) ABSTRACT

A single use needle-like probe contains optical fibers to deliver and collect light at the distal tip of the needle-like probe. The single use needle-like probe may connect to a handpiece that may contain sensors to monitor how the probe is being used. Sensors within the handpiece may, e.g., include a force sensor and a position sensor that detect the depth of the probe in tissue. The handpiece may be connected through a cable to a control unit that may include light sources, optical detectors, control electronics and one or more microprocessors to analyze the data collected.

This application is a continuation-in-part of U.S. patent application Ser. No. 10/803,574, titled “Optical Biopsy System With Single Use Needle Probe,” filed filed Mar. 17, 2004, incorporated herein by reference. This application claims priority to U.S. Provisional Patent Application Ser. No. 60/455,536, titled “Optical Biopsy System With Single Use Needle Probe,” filed Mar. 17, 2003, incorporated herein by reference. This application claims priority to U.S. Provisional Patent Application Ser. No. 60/760,196, titled “Tissue Probe With Speed Control,” filed Jan. 18, 2006, incorporated herein by reference.

### BACKGROUND OF THE INVENTION

1. Field of the Invention

The present invention relates to a tissue diagnostic probe and system that uses optical measurements of tissue to accurately determine tissue type or state.

2. Description of Related Art

Every week in the United States about 19,000 open surgical breast biopsies are performed on women. Only about 3000 cancers will be found. Thus, about 85% of the biopsies are unnecessary. This means about 16,000 women will needlessly undergo open surgical breast biopsies in the U.S. every week because of the inaccuracy in diagnosing cancerous tissue in the breast.

Open surgical breast biopsies are highly undesirable because they are invasive and traumatic to the patient. In a surgical biopsy, the suspected location of the abnormality would be marked with a thin, hooked guide-wire. The surgeon tracts the guide-wire to the location of the suspected abnormality and the suspect area is excised. The open surgical biopsy is the most common form of biopsy and is invasive, painful and undesirable to the patient. The open surgical biopsies may also leave scar tissue, which may obscure the diagnostic ability of future mammograms, creating a major handicap for the patient.

Another form of biopsy is a large-core needle biopsy (14 gauge needle). The standard core biopsies remove a 1 mm×17 mm core of tissue. The large core needle biopsy is less invasive than a surgical biopsy but still removes an undesirable amount of tissue.

Still another form of biopsy is the fine needle aspiration biopsy. In this type of biopsy, a small amount of the cells are aspirated for cytological analysis. This procedure is minimally invasive. A shortcoming, however, with fine needle aspiration is poor accuracy. The poor accuracy is a result of the small sample size, which makes accurate cytology difficult.

Another drawback of typical biopsy procedures is the length of time required for the laboratory to review and analyze the excised tissue sample. The wait can take, on average, approximately two months from the first exam to final diagnosis. Consequently, many women may experience intense anxiety while waiting for a final determination.

Various methods and devices have been developed to measure physical characteristics of tissue in an effort to distinguish between cancerous and non-cancerous tissue. For example, U.S. Pat. No. 5,303,026 to Strobl et al. (the Stroble patent) describes an apparatus and method for spectroscopic analysis of scattering media such as biological tissue. More specifically, the Stroble patent describes an apparatus and method for real-time generation and collection of fluorescence, reflection, scattering, and absorption information from a tissue sample to which multiple excitation wavelengths are applied.

U.S. Pat. No. 5,349,954 to Tiemann et al. also describes an instrument for characterizing tissue. The instrument includes, amongst other things a hollow needle for delivering light from a monochromator through the needle to a desired tissue region. Mounted in the shaft of the needle is a photodiode having a light sensitive surface facing outward from the shaft for detecting back-scattered light from the tissue region.

U.S. Pat. No. 5,800,350 to Coppleson et al. discloses an apparatus for tissue type recognition. In particular, an apparatus includes a probe configured to contact the tissue and subject the tissue to a plurality of different stimuli such as electrical, light, heat, sound, magnetic and to detect plural physical responses to the stimuli. The apparatus also includes a processor that processes the responses in combination in order to categorize the tissue. The processing occurs in real-time with an indication of the tissue type (e.g. normal, pre-cancerous/cancerous, or unknown) being provided to an operator of the apparatus.

U.S. Pat. No. 6,109,270 to Mah et al. and U.S. patent application Ser. No. 09/947,171 to Hular et al. disclose a multimodality instrument for tissue characterization. Although the multimodality probes described by Mah et al. and Hular et al. offer the potential of higher accuracy (i.e. sensitivity and specificity) the single use multimodality probes are expensive to produce.

Given the limitations of existing tissue biopsy techniques, there exists a need for an inexpensive, convenient and reliable single use probe that can provide real time analysis of tissue type and state. The present invention addresses this need.

### SUMMARY OF THE INVENTION

It is an object of the present invention is to provide a method and a system that can be used by physicians to accurately measure the optical properties of tissue over a wide wavelength range (typically 300 nm to 1000 nm).

It is another object of the invention to provide a system that can be used by surgeons to determine whether a suspicious lesion is cancer or normal tissue.

It is another object of the invention to provide a low cost single use probe and system that can be used by surgeons to quickly diagnose breast cancer.

It is an object of the present invention is to provide a method and a system that can be used by physicians to accurately measure the optical properties of tissue over a wide wavelength range.

It is another object of the invention to provide a system that can be used by surgeons to determine whether a suspicious lesion is cancer or normal tissue.

Some embodiments of the present invention include a single use needle-like probe that contains optical fibers to deliver and collect light at the distal tip of the needle-like probe. The single use needle-like probe may connect to a handpiece that may contain sensors to monitor how the probe is being used. Sensors within the handpiece may, e.g., include a force sensor and a position sensor that detect the depth of the probe in tissue. The handpiece may be connected through a cable to a control unit that may include light sources, optical detectors, control electronics and one or more microprocessors to analyze the data collected.

In another embodiment, the inner core of the needle-like probe contains an electrical conductor that along with the outer metal sheath comprises an electrode pair that can be used to measure the electrical properties of tissue over a broad frequency range (e.g., 1 KHz-1 MHz). Software within the control electronics analyzes the measured electrical properties and determines the type of tissue and possibly tissue state. The use of electrical properties to distinguish tissue type and state has been documented in numerous papers; a good review can be found in the following series of papers, all incorporated herein by reference: C. Gabriel, S. Gabriel, E. Corthout, The dielectric properties of biological tissues: I, Phys. Med. Biol. 41, 2231; S. Gabriel, R. W. Lau and C. Gabriel: The dielectric properties of biological tissues: II. Measurements in the frequency range 10 Hz to 20 GHz, Phys. Med. Biol. 41, 2251 (1996); S. Gabriel, R. W. Lau and C. Gabriel: The dielectric properties of biological tissues: III. Parametric models for the dielectric spectrum of tissues, Phys. Med. Biol. 41, 2271 (1996).

In normal use, the physician takes a new sterilized probe and connects it to the handpiece. The system is then activated and light exits the distal tip of the probe. The physician then inserts the probe into tissue and navigates it to the suspicious lesion. During the complete insertion the system measures the optical properties of the tissue, which can then be analyzed to determine tissue type and state.

The probe-to-handpiece connector may be keyed to only allow the probe to be connected in one orientation thereby aligning all the fibers optics. In one embodiment, the fiber optics within the handpiece and probe are proximity coupled. In an alternative embodiment, the handpiece contains optical lenses that couple light from/to the handpiece to/from the probe.

The control unit contains, e.g., white light sources to measure the absorption and scattering properties of tissue. A laser may be located within the control unit to excite tissue fluorescence. Grating spectrometers and filtered detectors may be within the control unit to measure the scattered light and fluorescence emission. A wide variety of sources and detectors may be used within the control unit and a good review of these can be found in “Tissue Optics: Applications in Medical Diagnostics and Therapy” SPIE MS102, Editor Valery V. Tuchin, incorporated herein by reference.

The handpiece may include sensors that can measure the force being applied on the probe to penetrate the tissue. This information can be used by the system to locate lesions, which are in many cases tougher than normal tissue. This is particularly the case for breast tissue. The handpiece may also includes a position sensor that can monitor the depth of the probe in tissue. In one embodiment, the position sensor connects to a slideable sheath that is coaxially disposed over the single use needle-like section of the probe.

In another variation of the present invention, optical fibers are coated with a reflective coating to reduce stray light from coupling between the fibers. An aluminum coating is a suitable coating.

Another variation of the present invention uses a light-absorbing polymer between the optical fibers to reduce stray light coupling between the fibers.

Another variation of the present invention includes a probe as described above wherein the probe further includes a memory device capable of storing useful information about the probe.

Another variation of the present invention includes a handpiece and cable that includes a reference optical fiber. The reference optical fiber extends from a controller, through a flexible cable connected to the handpiece, and into the handpiece. The reference optical fiber has a distal end and the distal end comprises a reflective coating to reflect light.

Another variation of the present invention includes the probe as described above wherein the probe further includes a single mode optical fiber to perform optical coherence domain reflectometry (OCDR).

Another variation of the present invention includes the probe as described above wherein the probe is sharp. In still another variation, the distal tip of the porbe is cut and polished at an angle less than 70 degrees and preferably ranging from 30 to 70 degrees.

Another variation of the present invention includes a probe having a plurality of fibers and electrical conductors. This variation may also feature a slideable sheath coaxially disposed over the needle like section of the probe. The sheath is retractable from the distal section as the probe is inserted into the tissue. This variation may also include a position sensor in the handpiece configured to read the position of the sheath relative to the distal tip of the probe.

Another variation of the present invention includes a method for identifying tissue comprising manually inserting a probe as recited in any one of the above-described probes.

Still another variation of the present invention is a tissue detection system comprising a single use needle-like probe with a plurality of optical fibers. The system also includes a handpiece with integrated force and position sensors, and a cable is connected to a control unit configured to deliver and collect light through the plurality of optical fibers.

Additional aspects and features of the invention will be set forth in part in the description which follows, and in part will become apparent to those skilled in the art upon examination of the following or may be learned by practice of the invention.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 illustrates the main components of an embodiment of the diagnostic system.

FIG. 2 shows a detailed cross sectional view through the center of a single use needle probe section.

FIG. 3 shows a variety of fiber optic configurations that can be integrated into a single use needle probe.

FIG. 4 shows a detailed cross sectional view through a handpiece.

FIG. 5 shows a detailed cross sectional view through an alternative embodiment of a handpiece.

FIG. 6 shows the measured optical spectrum for two different tissue types.

### DETAILED DESCRIPTION OF THE INVENTION

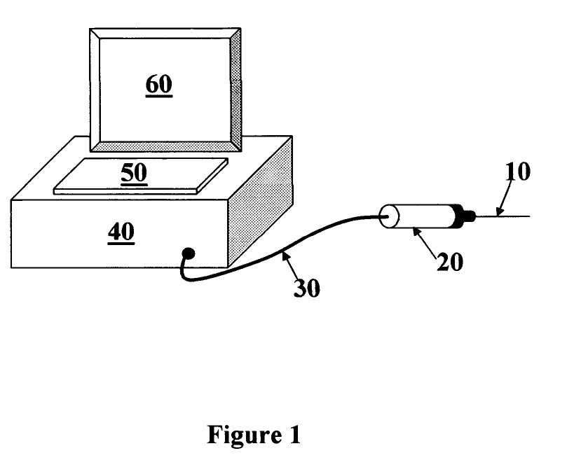

FIG. 1 shows the main components of the present invention. The single use needle-like probe 10 connects to handpiece 20 that is connected through a cable 30 to an electronic control unit 40. The control unit includes an input device 50 (e.g., a keyboard) and display 60 that provides the physician with information about the tissue near the tip of the probe 10. The probe 10 with integrated optical fibers emits and collects light near the distal tip, which light is measured and analyzed by the electronic control unit 40 to determine the tissue type and state. The cable 30 contains optical fibers and electrical wires.

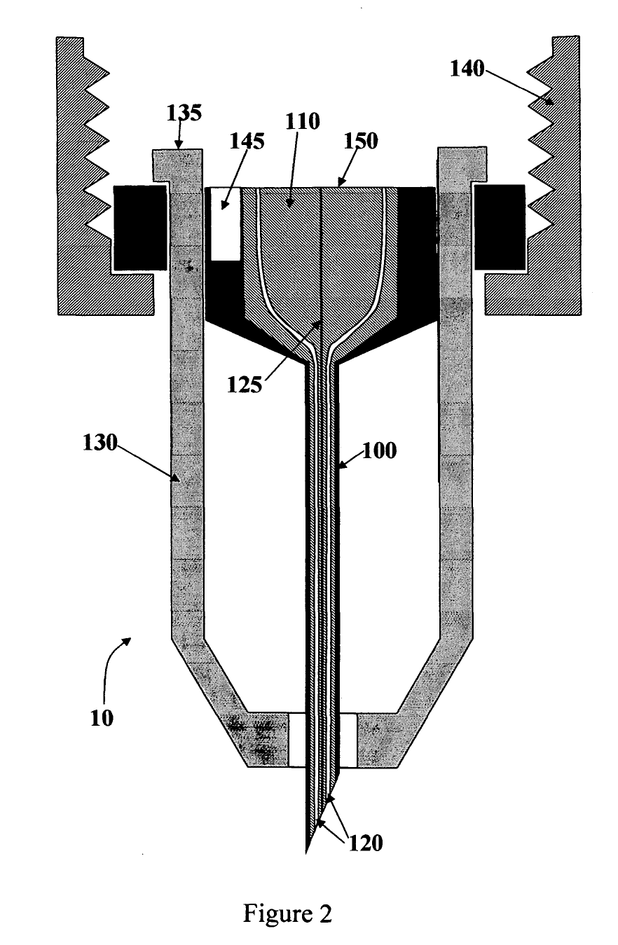

FIG. 2 shows a detailed cross sectional view through the center of the probe 10. The probe 10 is comprised of an outer metal sheath 100 that is bonded to an internal core 110 that contains the optical fibers 120. In one embodiment the probe contains a plurality of multimode optical fibers 120. In an alternative embodiment the probe contains a plurality of multimode and single mode optical fibers. An optional electrical conductor 125 can also be integrated into the internal core 110. The electrical conductor 125 when combined with the outer metal sheath 100 can be used to measure the electrical properties of the tissue.

A sliding sheath 130 is used to measure the depth of the probe in tissue. The sliding sheath 130 slides up and down the needle like section of the probe as it is inserted into tissue. When connected the proximal surface of the sheath 135 makes contact with a position sensor within the handpiece 20. The locking ring 140 is used to connect the probe 10 to the handpiece 20. An alignment key 145 insures that the probe 10 and handpiece 20 are properly aligned to achieve high coupling efficiency between the optical fibers. The surface 150 is polished and in one embodiment directly contacts the optical surface in the handpiece. The outer metal sheath 100 is similar to standard medical needles and is manufactured using techniques commonly known in the field. The inner core 110 is made of a biocompatible material (e.g., polyurethane, polyethylene, glass, ceramic). Biocompatible glues or epoxies are used to bond the optical fibers 120 inner core 110 and metal sheath 110 together.

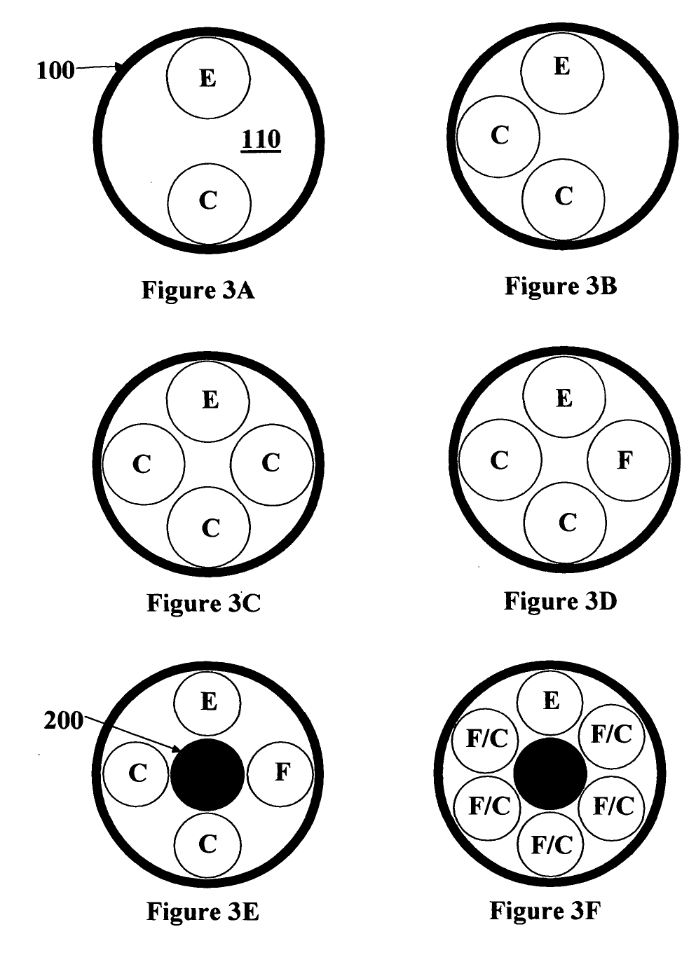

FIG. 3A-3E shows the distal tip of the probe 10 for a variety of fiber optic orientations. The simplest configuration shown in FIG. 3A has an outer metal sheath 100 and an inner core 110 with two imbedded multimode optical fibers. A fiber E is used to emit light and a second fiber C collects scattered light originally emitted by the first fiber E. FIG. 3B-3D shows configurations with multiple collection fibers, C, and a fluorescence fiber, F, that can emit and collect light simultaneously. Although the figures show all fibers with the same diameter it is possible to use different fiber sizes for each fiber. One of the fiber optics can also be a single mode fiber that can be used to perform optical coherence domain reflectometry. In an alternative embodiment, FIG. 3E, one of the optical fibers is replaced with an electrical conductor 200 to make measurements of the electrical properties of tissue. FIG. 3F, shows an alternative embodiment, where an electrical conductor 200 and multiple optical fibers (C, F, E) are integrated in the probe in a closed pack orientation. The electrical conductor 200 can be a single conducting wire, a coaxial cable, or multiple conducting wires.

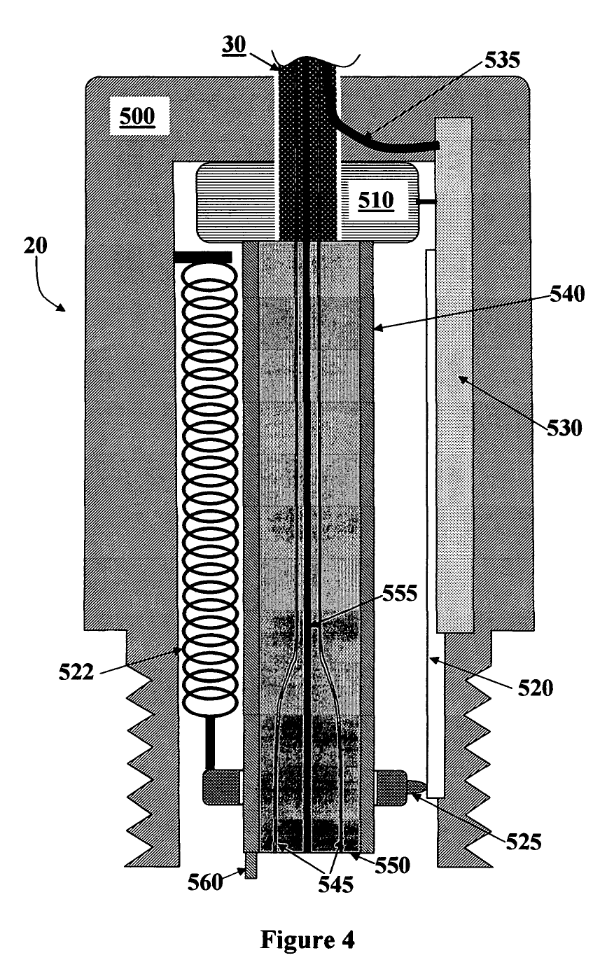

FIG. 4 shows a cross sectional view of the handpiece 20 showing the key components. An outer enclosure 500 encloses a force sensor 510, a position sensor 520, an electronics board 530, and a stiff shaft 540 with integrated fiber optics 545. A key 560 on the shaft mates to key opening 145 of the probe 10 (see FIG. 2) to properly align and connect the optical fibers 545 and electrical conductors 555 within the handpiece 20 to the optical fibers 120 and electrical conductors 125 within the probe 10. The surface of the docking tip 550 is polished to improve light coupling between the handpiece 20 and the probe 10. In one embodiment the surface of the docking tip 550 and the probe surface 150 are polished at an angle (e.g. 8 degrees) to reduce back reflections. The force sensor 510 measures the force applied at the distal end of the shaft 540. A wide variety of force sensors exist that can be integrated into the handpiece (e.g., strain gauge, tactile sensors, piezoelectric force sensors). The position sensor 520 measures the position of the sliding ring 525 that is moved by the sliding sheath 130 that is integrated into the probe 10. A spring 522 connected to the sliding ring 525 maintains contact between the sliding ring 525 and the sliding sheath 130. A wide variety of position sensors exist that can be integrated into the handpiece (e.g., potentiometric sensors, optical sensors, capacitive sensors). A description of suitable sensors can be found in “Handbook of Modern Sensors: physics, designs, and applications” 2^{nd} edition by Jacob Fraden, incorporated herein by reference. The electronics board 530 conditions the force sensor 510 and position sensor 520 signals and transmits them through wires 535 that integrate into cable 30. In one embodiment the electronics board 530 includes an analog to digital converter and the measurements are transmitted as digital values.

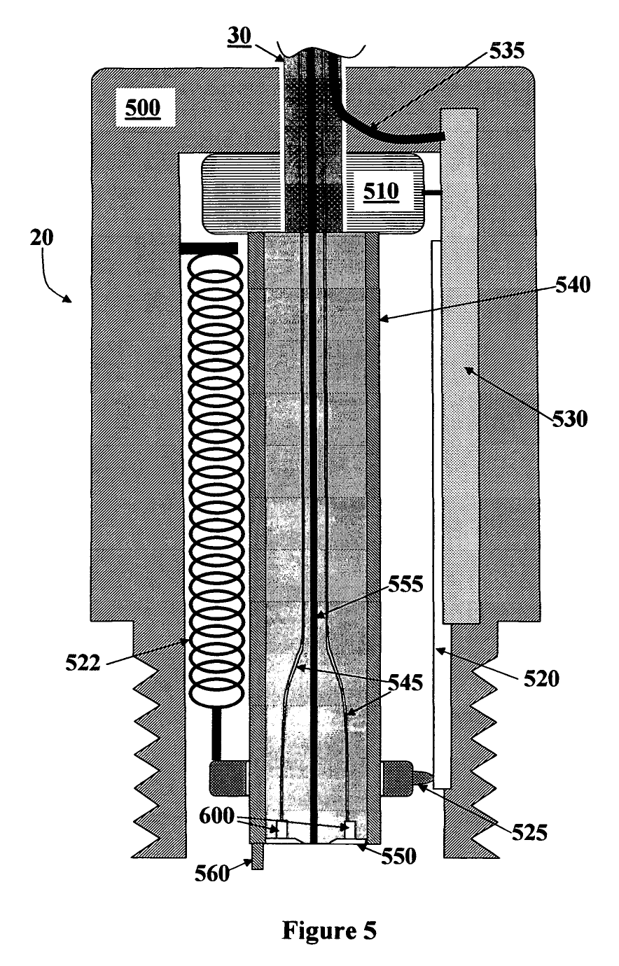

FIG. 5 shows a cross sectional view of an alternative handpiece 20 showing the key components. In this embodiment grin lens 600 integrated into the handpiece shaft 540 couple the light between the handpiece fiber optics 545 and probe 10 fiber optics 120. When the handpiece 20 and probe 10 are connected an air gap between the grin lens and the probe fiber optics reduces the risk of damaging the optical surface when the connection is made.

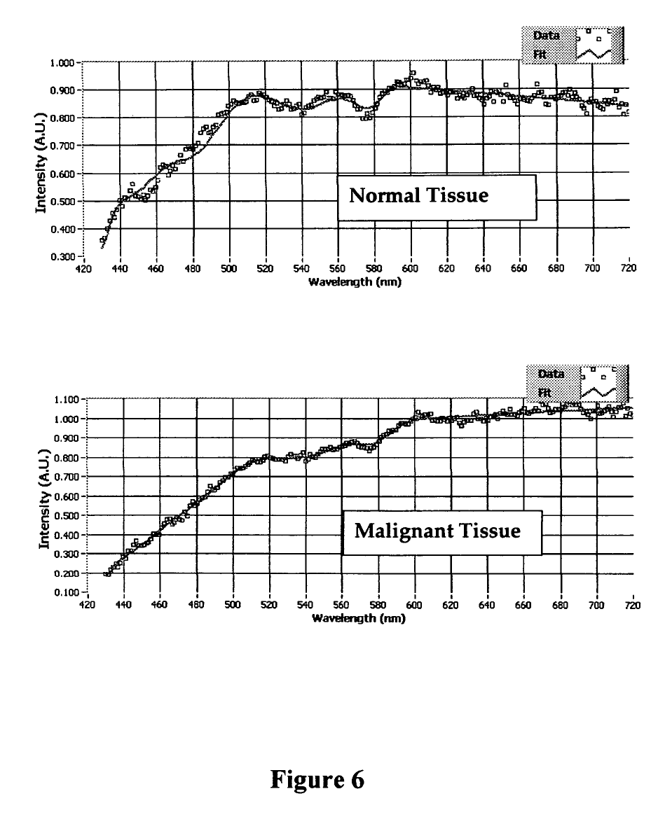

FIG. 6 shows the measured optical spectrum for normal and malignant breast tissue. A needle-like probe with one emission and one collection fiber was used to acquire this data. The absorption feature between 520 nm and 600 nm is due to blood absorption.

Applications for the present invention can vary widely. For example, the present invention may be used to detect cancerous tissue in the breast. The probe of the present invention may also be used to characterize other types of abnormalities found in other locations of the body. The probe of the present invention may be used in vivo as described above or alternatively, the probe may be used to identify tissue in vitro. Preferably, the probe of the present invention is configured to measure tissue properties in real-time and continuously as the probe tip is inserted into a tissue sample. Signals from the multiple sensors of the probe are immediately processed to quickly diagnosis, identify or characterize the tissue.

The device of the present invention may also be used in combination with other medical devices. For example, the probe may be inserted through a cannula or other tubular structure used in medical procedures.

All of the features disclosed in the specification (including any accompanying claims, abstract and drawings), and/or all of the steps of any method or process disclosed, may be combined in any combination, except combinations where at least some of such features and/or steps are mutually exclusive. Each feature disclosed, in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiments. The invention extends to any novel one, or any novel combination, of the features disclosed in this specification (including any accompanying claims, abstract and drawings), or to any novel one, or any novel combination, of the steps of any method or process so disclosed.

I claim:

1. A diagnostic system for determining the properties of a sample, comprising:
a probe that includes:
a probe shaft comprising a proximal end and a distal end, wherein said proximal end is mounted to a support that comprises a key-opening;
at least one first fiber optic that traverses the length of the inside of said probe shaft;
a first core material holding said at least one first fiber optic in place within said probe shaft; and
a first connector element fixedly attached to said support; and
a handle that includes:
a handle shaft having a proximal end and a distal end;
at least one second fiber optic traversing the length of the inside of said handle shaft;
a second core material holding said at least one second fiber optic in place within said handle shaft;
a key fixedly attached to said handle shaft, wherein when said key is inserted into said key-opening, said at least one first fiber optic is in optical alignment with said at least one second fiber optic; and
a second connector element fixedly attached to said handle shaft, wherein said first connector element and said second connector element are connectable together.

2. The system of claim 1, wherein said support further comprises a sheath passageway, wherein said system further comprises:
a slidable sheath over said cylindrical shaft, said sheath having an opening at its distal end for passage of a portion of said probe shaft, wherein said slidable sheath is positioned to slide through said sheath passageway;
a sliding piece slidably mounted to said handle shaft, wherein said sliding piece can slide from a starting position relatively near said distal end of said shaft to an ending position relatively near said proximal end of said shaft;
means for exerting a biasing force on said sliding piece that biases said sliding piece to remain at said starting position in the absence of a counter force greater than said biasing force, wherein said slidable sheath comprises a sheath proximal surface that contacts said sliding piece when said key is inserted into said key-opening, wherein when said probe shaft is inserted into a testing medium, said sheath will exert said counter force and move said sliding piece in proportion to the depth that said probe shaft enters said testing medium; and
a position sensor positioned to sense the position of said sliding ring.

3. The system of claim 2, wherein said sliding piece comprises a sliding ring.

4. The system of claim 2, wherein said means for exerting a biasing force comprises a spring.

5. The system of claim 2, wherein said position sensor is selected from the group consisting of a potentiometric sensor, an optical sensor and a capacitive sensor.

6. The system of claim 1, wherein said probe shaft comprises a cylinder, wherein said distal end is sharpened.

7. The system of claim 1, wherein said probe shaft comprises an electrically conductive material.

8. The system of claim 1, wherein said probe shaft comprises a hypodermic needle.

9. The system of claim 1, wherein said at least one first fiber optic or said at least one second fiber optic are selected from the group consisting of a single mode fiber optic and a multimode fiber optic.

10. The system of claim 1, wherein said handle shaft comprises a cylinder.

11. The system of claim 1, wherein said first core material and said second core material comprise the same material.

12. The system of claim 1, wherein said first core material and said second core material comprise biocompatible material.

13. The system of claim 12, wherein said biocompatible material is selected from the group consisting of polyurethane, polyethylene, glass and ceramic.

14. The system of claim 1, further comprising biocompatible glue or epoxy as a bonding agent to bond together said at least one first optical fiber, said first core material and said probe shaft.

15. The system of claim 1, further comprising a force sensor operatively connected to said handle shaft to measure the force applied at the distal end of said handle shaft.

16. The system of claim 15, wherein said force sensor is selected from the group consisting of a strain gauge, a tactile sensor and a piezoelectric force sensor.

17. The system of claim 1, further comprising a control unit connected to said handle by a cable that includes at least one third fiber optic operatively connected to said at least one second fiber optic, wherein said control unit includes an input device, a display, a light source, means for inputting light from said light source into said at least one third fiber optic, means for collecting light that returns through said at least one third fiber optic to said control unit and means for analyzing the collected light to determine a property of said sample.

18. The system of claim 17, wherein said at least one second fiber optic comprises a length of fiber optic within said handle shaft, wherein said at least one third fiber optic comprises a length of fiber optic within said cable, wherein said length of fiber optic within said handle shaft together with said length of fiber optic within said cable comprise at least one integral, undivided length of fiber optic.

19. The system of claim 1, wherein said at least one second fiber optic comprises a grin lens at its distal end.

20. The system of claim 1, wherein said probe shaft further comprises at least one first electrical conductor that traverses the length of said probe shaft, wherein said handle shaft further comprises at least one second electrical conductor that traversed the length of said handle shaft, wherein said at least one first electrical conductor will be in electrical contact with said at least one second electrical conductor when said key is inserted into said key-opening.

21. The system of claim 20, further comprising a control unit connected to said handle by a cable that includes at least one third fiber optic operatively connected to said at least one second fiber optic, wherein said control unit includes an input device, a display, a light source, means for inputting light from said light source into said at least one third fiber optic, means for collecting light that returns through said at least one third fiber optic to said control unit and means for analyzing the collected light to determine a property of said sample, wherein said control unit further includes a source of electrical energy, means for inputting said electrical energy from said source of electrical energy into said at least one second electrical conductor, means for collecting electrical energy and means for analyzing the collected electrical energy to determine a property of said sample.

22. The system of claim 17, further comprising an electronics board operatively fixed within said handpiece to organize a signal from said sliding piece and communicate said signal to said control unit.

23. The system of claim 17, further comprising a force sensor operatively connected to said handle shaft to measure the force applied at the distal end of said handle shaft, further comprising an electronics board operatively fixed within said handpiece to organize a signal from said force sensor and communicate said signal to said control unit.

24. The system of claim 17, further comprising a reference fiber optic that extends from said control unit through said cable and into said handpiece, wherein said reference fiber optic has a coated distal end comprising a reflective coating.
